(19) United States
(12) Patent Application Publication
(10) Pub. No.: US 2017/0072209 A1
(43) Pub. Date: Mar. 16, 2017

(54) HIGH-FREQUENCY THERMOTHERAPY DEVICE

(71) Applicant: JS ON CO.,LTD, Songpa-gu Seoul (KR)

(72) Inventor: Jung Su KIM, Ansan-si (KR)

(21) Appl. No.: 15/101,420

(22) PCT Filed: Jan. 27, 2015
(86) PCT No.: PCT/KR2015/000819

§ 371 Date: Jun. 3, 2016

(30) Foreign Application Priority Data
Apr. 17, 2014 (KR) 10-2014-0045839

(51) Int. Cl. A61N 1/40; A61N 1/06; A61B 5/053

(52) U.S. Cl. CPC A61N 1/403; A61B 5/053; A61N 1/06

(57) ABSTRACT

The present disclosure provides a high-frequency thermotherapy device comprising: an impedance sensing module configured to measure impedances of normal and abnormal muscles; a control module configured to receive the measurement impedance from the impedance sensing module and to determine whether the abnormal muscle is detected; a frequency and output adjustment module configured to automatically generate a frequency adjustment signal to enable a about 2 to 5 MHz frequency to be generated upon determination that the abnormal muscle is detected.

### CROSS-REFERENCE TO RELATED APPLICATION

This application claims the benefit of Korea Patent Application No. 10-2014-0045839 filed on Apr. 17, 2014, the entire content of which is incorporated herein by reference for all purposes as if fully set forth herein.

### BACKGROUND

Field of the Present Disclosure

The present disclosure relates to high-frequency thermotherapy device. More particularly, the present disclosure relates to a high-frequency thermotherapy device including an impedance sensing function to measure impedances of normal and abnormal muscles, wherein the normal and abnormal muscles indicate different impedance values; a determination function to determine whether the abnormal muscle is detected based on the measurement impedances; a frequency and output adjustment function to automatically generate a frequency adjustment signal to enable a about 2 to 5 MHz frequency to be generated upon determination that the abnormal muscle is detected, whereby, using the device, the abnormal muscle is healed while the normal muscle is not affected, and a pain killer is less consumed; wherein a display module of the device includes control panel configured to allow an operator of the device to set a timer, a voltage level, and/or an output level including high, middle, low levels, whereby the operator may manipulate the device in a more convenient manner; wherein the display module has a LED configured to change its brightness based on the output level and to turn on/off based on a presence/absence of the output, whereby the operator may easily perceive the operation state of the device; wherein a hand-piece is configured to generate the deep heat via a ceramic member disposed therein, based on a timing schedule to suppress a risk of a skin burn even though the ceramic member contacts the skin in a long time; and/or wherein the device is connected over a wired or wireless network to a computer to allow a remote adjustment of the output and to monitor or regulate the control module.

Discussion of the Related Art

Recently, a physical therapy device such as a helium lamp, a infrared lamp, etc. has been employed in a home, a specific place, etc. One kind of the physical therapy device is a high-frequency thermotherapy device to apply a heat to an abnormal portion of the body of the user.

The high-frequency thermotherapy device is used to apply high-frequency RF energy to the human body which, in turn, is transformed to a heat energy in the human body. The heat energy increases a temperature of the human body to function as a medical treatment or a skin care.

In this connection, the term “high-frequency” refers to a radio transmission frequency 300 kHz to 1,200 kHz in a dictionary sense. However, in the medical sense, the term “high-frequency” may refer to a RF high-frequency current.

A general commercial alternating current may have a 60 Hz frequency. In the medical field, 0.3 MHz to several tens MHz frequencies may be employed.

When the high-frequency AC current is applied to the human body, ions in cells in the body may alternately change in a polarity thereof, to generate frictional heat energy to work as a medical treatment or skin care.

The frictional heat energy may have a temperature of average 35° C. to 45° C. to be transferred into the deep portion of the body. This heat energy may be applied continuously and repeatedly to the deep portion of the body to enlarge a width of the blood vessel. This may lead to activation of the metabolism and thus a self-healing of the human body.

The hot heat energy may have sterilization and generation of collagens in a dermis of the skin. The hot heat energy may relax the stressed muscle tissue. The hot heat energy may have various effects such as pain killing, anti-wrinkling, loss of weight, skin pore size reduction, etc.

Generally, the high-frequency thermotherapy may have pads including therein ceramic members to generate heat energy. In use, the ceramic members tightly contact the abnormal site of the body to heal the same.

In this connection, the plurality of the ceramic members may operate simultaneously. Further, during the treatment, the operator of the device may not be aware of the temperatures of the plurality of the ceramic members. Thus, when the plurality of the ceramic members tightly contacts the body site in a long time, it may have a risk that the human body may burn.

In addition to the RF high-frequency energy, a variety of energies may be applied to the human skin to change a skin tissue state, or improve a skin tissue characteristic or heal the abnormal skin. The variety of energies may include, for example, laser beam energy, flash lamp energy, ultrasonic energy, etc. Especially, the skin treatment device using the RF high-frequency wave energy has been studied vigorously.

When the high-frequency energy is applied to the skin, molecules in the skin tissue may vibrate, rotate, twist, collide with each other via alternate change of current directions, thereby to cause the frictions between the molecules, and, in turn, heat energy deeply in tissue, This deep heat may increase the temperature of the skin tissue, to re-organize the collagen layer. This may lead to anti-wrinkling and restored skin elasticity.

Further, the deep heat may enhance a blood flow in the skin tissue to lead to anti-aging of the skin.

Korean patent application laid-open number 10-2010-0101420 (published on Sep. 17, 2010) discloses a high-frequency thermotherapy device.

Using the high-frequency thermotherapy device, there may occur different effects based on skin sites having the high-frequency energy applied thereto.

Therefore, an optimal treatment effect may be achieved by applying the energy into different skin sites based on treatment purposes. However, the high-frequency thermotherapy device as disclosed in the above patent document may lack the multi-purposes uses.

In the high-frequency thermotherapy device as disclosed in the above patent document, there are not present following functions: normal and abnormal muscles are detected based on impedance measurements thereof by the impedance sensor; a high-frequency current generation module automatically generates a current with 2 to 5 MHz frequency upon determination that the abnormal muscle is detected whereby the normal muscle is not affected in a negative manner, but only the abnormal muscle is healed. Further, in the high-frequency thermotherapy device as disclosed in the above patent document, there are not present following functions: the output level is adjustment to a high, middle or low level based on the state of the abnormal potion the body, and the timing schedule for the application of the RF energy is set to prevent the skin burn.

Thus, there is a need for a novel high-frequency thermotherapy device with the following functions: normal and abnormal muscles are detected base d on impedance measurements thereof by the impedance sensor, a high-frequency current generation module automatically generates a current with 2 to 5 MHz frequency upon determination that the abnormal muscle is detected, whereby the normal muscle is not affected in a negative manner, but only the abnormal muscle is healed; and/or with the following functions: the output level is adjustment to a high, middle or low level based on the state of the abnormal potion the body, and the timing schedule for the application of the RF energy is set to prevent the skin burn.

### SUMMARY

From considerations of the above situations, the present disclosure provides a high-frequency thermotherapy device including an impedance sensing function to measure impedances of normal and abnormal muscles wherein the normal and abnormal muscles indicate different impedance values, a determination function to determine whether the abnormal muscle is detected based on the measurement impedances; a frequency and output adjustment function to automatically generate a frequency adjustment signal to enable a about 2 to 5 MHz frequency to be generated upon determination that the abnormal muscle is detected, whereby, using the device, the abnormal muscle is healed while the normal muscle is not affected and a pain killer is less consumed.

The present disclosure further provides a high-frequency thermotherapy device including a display module having a control panel configured to allow an operator of the device to set a timer, a voltage level, and/or an output level including high, middle, low levels, whereby the operator may manipulate the device in a more convenient manner.

The present disclosure further provides a high frequency thermotherapy device including a LED configured to change its brightness based on the output level and to turn on/off based on, a presence absence of the output, whereby the operator may easily perceive the operation state of the device.

The present disclosure further provides a high frequency thermotherapy device including a hand-piece configured to generate the deep heat via a ceramic member disposed therein, based on a timing schedule to suppress a risk of a skin burn even though the ceramic member contacts the skin in a long time.

The present disclosure further provides a high-frequency thermotherapy device con netted over a wired or wireless network to a computer to allow a remote adjustment of the output and to monitor or regulate a control module.

In one aspect, the present disclosure provides a high-frequency thermotherapy device comprising: a main body; a display module disposed on a top portion of the main body, wherein the display module is configured to display a current operation state, a set output level including high, middle, low levels, a set voltage level, and/or a use time; a main power supply disposed below the display module; an impedance sensing module configured to measure impedances of normal and abnormal muscles, wherein the normal and abnormal muscles indicate different impedance values; a control module configured to receive the measurement impedance from the impedance sensing module and to determine whether the abnormal muscle is detected based on the measurement impedances; a frequency and output adjustment module being connected to the control module and including a frequency adjustment sub-module and an output adjustment sub-module, wherein the frequency adjustment sub-module is configured to automatically generate frequency adjustment signal to enable a about 2 to 5 MHz frequency to be generated upon determination that the abnormal muscle is detected, wherein the output adjustment sub-module is configured to automatically generate an output adjustment signal to enable a about 100Ω to 1000Ω output within 500 W to be generated upon determination that the abnormal muscle is detected; a high-frequency current generation module including a high-frequency current generation sub-module and an electrode sub-module, wherein the high-frequency current generation sub-module is connected to the main power supply and the frequency and output adjustment module, wherein the high-frequency current generation sub-module is configured to receive the frequency and output adjustment signals and to generate a high-frequency current based the frequency and output adjustment signals, and the electrode sub-module is configured to receive, the generated high-frequency current from the high-frequency current generation sub-module; and a hand-piece connected to the electrode sub-module, and including therein a ceramic member, wherein the hand-piece is configured to receive the generated high-frequency current and to generate a deep heat using the ceramic member and to apply the deep heat to the abnormal muscle, wherein the hand-piece is configured to generate the deep heat based on a timing schedule set by the control module.

In one embodiment, the display module may comprise: a control panel configured to allow an operator of the device to set a timer, a voltage level, and/or an output level including high, middle, low levels; a LED configured to change its brightness based on the output level and to turn on/off based on a presence/absence of the output; and an auxiliary power supply configured to supply a power to the control panel and the LED.

In one embodiment, the output adjustment sub-module may be configured to generate the output adjustment signal to selectively enable the outputs 70V, 80V, and 98V to be generated respectively for a skin care, an anti-inflammation and a malignant inflammation treatment.

In one embodiment, the device may be connected over a wired or wireless network to a computer to allow a remote adjustment of the output and to monitor or regulate the control module.

The high-frequency thermotherapy device as described above may have following effects:

First, the present high-frequency thermotherapy device has an impedance sensing function to measure impedances of normal and abnormal muscles, wherein the normal and abnormal muscles indicate different impedance values, a determination function to determine whether the abnormal muscle is detected based on the measurement impedances; a frequency and output adjustment function to automatically generate a frequency adjustment signal to enable a about 2 to 5 MHz frequency to be generated upon determination that the abnormal muscle is detected, whereby, using the device, the abnormal muscle is healed while the normal muscle is not affected and a pain killer is less consumed.

Second, the present high-frequency thermotherapy device has the display module having the control panel configured to allow the operator of the device to set the timer, voltage level, and/or output level including high, middle, low levels, whereby the operator may manipulate the device in a more convenient manner.

Third, the present high-frequency thermotherapy device has the LED configured to change its brightness based on the output level and to turn on/off based on a presence/absence of the output, whereby the operator may easily perceive the operation state of the device.

Fourth, the present high-frequency thermotherapy device has the hand-piece configured to generate the deep heat via the ceramic member disposed therein, based on the timing schedule set by the control module, to suppress a risk of a skin burn even though the ceramic member contacts the skin in a long time.

Fifth, the present high-frequency thermotherapy device is connected over a wired or wireless network to a computer to allow a remote adjustment of the output and to monitor or regulate the control module.

### BRIEF DESCRIPTIONS OF THE DRAWINGS

FIG. 1 shows a perspective view of an appearance of a high-frequency thermotherapy device in accordance with one embodiment of the present disclosure.

FIG. 2 illustrates a schematic block diagram of a configuration of a high-frequency thermotherapy device in accordance with one embodiment of the present disclosure.

### DETAILED DESCRIPTIONS

Hereinafter, a preferred embodiment of the present disclosure will be described in details with reference to attached drawings. Unless otherwise defined, all terms including technical and scientific terms used herein have the same meaning as commonly understood by one of ordinary skill in the art to which this inventive concept belongs. In the following description numerous specific details are set forth in order to provide a thorough understanding of the present disclosure. The present disclosure may be practiced without some or all of these specific details. In other instances, well-known process structures and/or processes have not been described in detail in order not to unnecessarily obscure the present disclosure.

Hereinafter, a high-frequency thermotherapy device in accordance with one embodiment of the present disclosure will be described in details with reference to attached drawings.

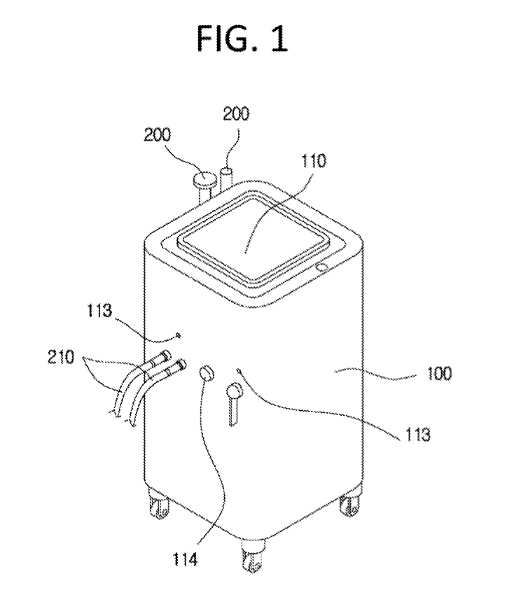

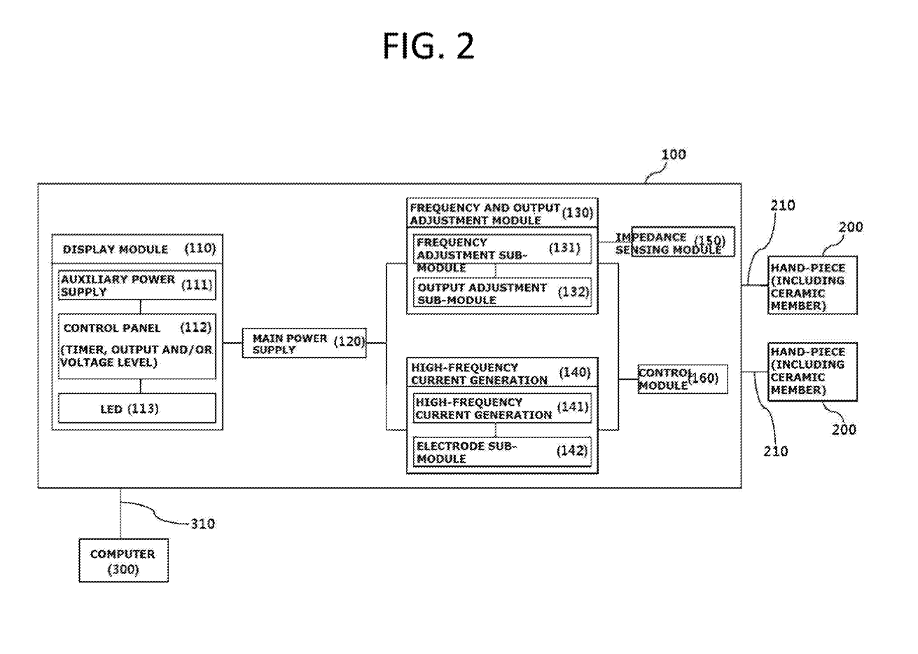

FIG. 1 shows a perspective view of an appearance of a high-frequency thermotherapy device in accordance with one embodiment of the present disclosure. FIG. 2 illustrates a schematic block diagram of a configuration of a high-frequency thermotherapy device in accordance with one embodiment of the present disclosure.

As shown in FIG. 1 and FIG. 2, a high-frequency thermotherapy device in accordance with one embodiment of the present disclosure may include a main body 100; a display module 110 disposed on a top portion of the main body 100, wherein the display module 110 is configured to display a current operation state, a set output level including high, middle, low levels, a set voltage level, and/or a use time; a main power supply 120 disposed below the display module 110; an impedance sensing module 150 configured to measure impedances of normal and abnormal muscles, wherein the normal and abnormal muscles indicate different impedance values; a control module 160 configured to receive the measurement impedance from the impedance sensing module 150 and to determine whether the abnormal muscle is detected based on the measurement impedances; a frequency and output adjustment module 130 being connected to the control module 160 and including a frequency adjustment sub-module 131, and an output adjustment sub-module 132, wherein the frequency adjustment sub-module 131 is configured to automatically generate a frequency adjustment signal to enable a 2 to 5 MHz frequency to be generated upon determination that the abnormal muscle is detected, wherein the output adjustment sub-module 132 is configured to automatically generate an output adjustment signal to enable a 100Ω to 1000Ω output within 500W to be generated upon determination that the abnormal muscle is detected; a high-frequency current generation module 140 including a high-frequency current generation sub-module 141 and an electrode sub-module 142, wherein the high-frequency current generation sub-module 141 is connected to the main power supply 120 and the frequency and output adjustment module 130, wherein the high-frequency current generation sub-module 141 is configured to receive the frequency and output adjustment signals and to generate a high-frequency current based the frequency and output adjustment signals, and the electrode sub-module 142 is configured to receive the generated high-frequency current from the high-frequency current generation sub-module 141; and a hand-piece 200 connected to the electrode sub-module 142, and including therein a ceramic member, wherein the hand-piece 200 is configured to receive the generated high-frequency current and to generate a deep heat using the ceramic member and to apply the deep heat to the abnormal muscle, wherein the hand-piece 200 is configured to generate the deep heat based on a timing schedule set by the control module 160.

Hereinafter, the above described components or modules of the present high-frequency thermotherapy device will be described below in>details in terms of a configuration and function.

The main body 100 of the high-frequency thermotherapy device may contain therein or thereon the display module 110, the main power supply 120, the frequency and output adjustment module 130, the high-frequency current generation module 140, and the control module 160.

The display module 110 may be disposed on a top portion of the main body 100. The display module 110 may be configured to display a current operation state, a set output level including high, middle, low levels, a set voltage level, and/or a use time. In this way, the operator of the high-frequency thermotherapy device may be informed of the current operation state, set output level, set voltage level, and/or use time.

In this connection, the display module 110 may include control panel 112 configured to allow the operator to set a timer, the voltage level, and/or the output level including high, middle, low levels. The display module 110 may include a LED 113 configured to change its brightness based on the output level and to turn on/off based on a presence/absence of the output. The display module 110 may include an auxiliary power supply 111 configured to supply a power to the control panel 112 and the LED 113. A left side LED 113 on a front side of the main body as shown in FIG. 1 may be configured to change, in a color based on the measurement impedance when the ceramic member contacts the skin of the patient. In this way, the operator may recognize a current treatment situation. A right side LED 113 on a front side of the main body as shown in FIG. 1 may be configured to turn on/off based on on/off of the main power supply.

The main power supply 120 may be disposed below the display module 110. The main power supply 120 may be configured to supply the power to the high-frequency current generation sub-module 141 to generate the high-frequency current.

The impedance sensing module 150 may be attached to the hand-piece 200. The impedance sensing module 150 may be configured to measure impedances of normal and abnormal muscles, wherein the normal and abnormal muscles indicate different impedance values. The control module 160 may be configured to receive the measurement impedance from the impedance sensing module 150 and to determine whether the abnormal muscle is detected. For this, the control module 160 may store therein an impedance relationship between the normal and abnormal muscles. The control module 160 may be configured to control the time schedule of the deep heating by the ceramic member.

The frequency and output adjustment module 130 may be connected to the control module 160. The module 130 may include the frequency adjustment sub-module 131, and the output adjustment sub-module 132. The frequency adjustment sub-module 131 may be configured to automatically generate the frequency adjustment signal to enable a frequency corresponding to the treatment of the abnormal muscle to be generated upon determination that the abnormal muscle is detected.

The output adjustment sub-module 132 may be configured to automatically generate the output adjustment signal to enable an output corresponding to the treatment of the abnormal muscle to be generated upon determination that the abnormal muscle is detected.

In this connection, the frequency corresponding to the treatment of the abnormal muscle may be an about 2 to 5 MHz frequency. The output corresponding to the treatment of the abnormal muscle may be a about 100Ω to 1000Ω output within 500 W.

Further, the output adjustment sub-module 132 may be configured to generate the output adjustment signal to enable an output 70V to be generated when the present thermotherapy device has an application of a skin care, The output adjustment sub-module 132 may be configured to generate the output adjustment signal to enable an output 80V to be generated when the present thermotherapy device has an application of an anti-inflammation. The output adjustment sub-module 132 may be configured to generate the output adjustment signal to enable an output 98V to be generated when the present thermotherapy device has an application of a treatment for a malignant inflammation. As shown in FIG. 1, a knob 114 at a middle portion of the front of the main body may be configured to be turned around to adjust the voltage to 70V, 80V and 98V on a 10V to 18V basis. Further, the operator may turn around the knob to adjust the voltage between 70V and 80V to 73V (low), 76V (middle), 79V (high), that is, in a 3V basis. The present disclosure is not limited thereto. The voltage difference between the low, middle, and high levels may vary depending on a choice of the operator.

The high-frequency current generation module 140 may include the high-frequency current generation sub-module 141 and the electrode sub-module 142.

The high-frequency current generation sub-module 141 may be connected to the main power supply 120 and the frequency and output adjustment module 130. The high-frequency current generation sub-module 141 may be configured to receive the frequency and output adjustment signals and to generate a high-frequency current based the frequency and output adjustment signals. The electrode sub-module 142 may be configured to receive the generated high-frequency current from the high-frequency current generation sub-module 141.

Using the high-frequency current generation module 140, the following treatment may be available. An inflammation (including malicious tumor) occurring in an abnormal portion of the body may start to be eradicated by application of 42° C. heat, and may be entirely eradicated by application of at least 45° C. heat. In this way, the high-frequency thermotherapy using the present device may be repeated to remove the abnormal portion (including malicious tumor) of the body. This treatment may have synergy effect together with use of chemical medical treatment. However, only the high-frequency thermotherapy using the present device may be remarkably effective. An effective treatment duration may be 5 to 30 minutes depending on the abnormal portion.

The hand-piece 200 may be connected to the electrode sub-module 142 of the main body 100 via the cable 210. The hand-piece 200 may include therein the ceramic member (not shown) which may generate a deep heat which in turn may be transferred to the abnormal portion of the body. Thus, the ceramic member contacts the skin of the body in use. The ceramic member may be disposed on a free end of the hand-piece 200.

In this connection, the hand-piece 200 may be classified into a contact-type hand-piece where the piece contacts the skin, and an invasive-type hand-piece where the piece penetrates into the muscle. In one embodiment, each of the contact-type handpiece and the invasive-type hand-piece may be single or plural. The contact-type and-piece and the invasive-type hand-piece may be combinational. For example, only a plurality of the contact-type hand-pieces may be connected to the main body. Only a plurality of the invasive-type hand-pieces may be connected to the main body. A combination of a single contact-type hand-piece and a single invasive-type hand-piece may be connected to the main body.

The hand-piece 200 may be configured to receive the generated high-frequency current from the high-frequency current generation module 140 and to generate a deep heat using the ceramic member and to apply the deep heat to the abnormal muscle. The hand-piece 200 may be configured to generate the deep heat based on the timing schedule set by the control module 160.

The high-frequency thermotherapy device as described above may be connected over a wired or wireless network 310 to a computer 300 to replace the manual manipulation of the knob with a remote manipulation, and/or monitor or regulate the control module 160. This may achieve a remote control of the present high-frequency thermotherapy device.

Regarding actual practice of the high-frequency thermotherapy device as described above, a patient is laid on a treatment table, and the hand-piece 200 is placed on the abnormal site of the patient, and a heat is applied to the site together with the high-frequency current with the varying 2 to 5 MHz high-frequency.

In this way, the inflammation of the abnormal muscle or skin may be removed by the non-invasive energy (42° C. or above) application only into the abnormal site, This may lead to direct inflammation eradication. Further, metabolism and immune system may be activated.

The above description is not to be taken in a limiting sense, but is made merely for the purpose of describing the general principles of exemplary embodiments, and many additional embodiments of this disclosure are possible. It is understood that no limitation of the scope of the disclosure is thereby intended. The scope of the disclosure should be determined with reference to the Claims. It is intended to cover alternatives, modifications, and equivalents as may be included within the spirit and scope of the present disclosure as defined by the appended claims.

The high-frequency thermotherapy device in accordance with the present disclosure may have various applications including the skin care, and the inflammation removal, etc.

What is claimed is:

1. a high-frequency thermotherapy device comprising:
a main body;
a display module disposed on a top portion of the main body, wherein the display module is configured to display a current operation state, a set output level including high, middle, low levels, a set voltage level, and/or a use time;
a main power supply disposed below the display module;
an impedance sensing module configured to measure impedances of normal and abnormal muscles, wherein the normal and abnormal muscles indicate different impedance values;
a control module configured to receive the measurement impedance from the impedance sensing module and to determine whether the abnormal muscle is detected based on the measurement impedances;
a frequency and output adjustment module being connected to the control module and including a frequency adjustment sub-module and an output adjustment sub-module, wherein the frequency adjustment sub-module is configured to automatically generate a frequency adjustment signal to enable a about 2 to 5 MHz frequency to be generated upon determination that the abnormal muscle is detected wherein the output adjustment sub-module is configured to automatically generate an output adjustment signal to enable a about 100Ω to 1000Ω output within 500 W to be generated upon determination that the abnormal muscle is detected;
a high-frequency current generation module including a high-frequency current generation sub-module and an electrode sub-module, wherein the high-frequency current generation sub-module is connected to the main power supply and the frequency and output adjustment module, wherein the high-frequency current generation sub-module is configured to receive the frequency and output adjustment signals and to generate a high-frequency current based the frequency and output adjustment signals, and the electrode sub-module is configured to receive the generated high-frequency current from the high-frequency current generation sub-module; and
a hand-piece connected to the electrode sub-module, and including therein a ceramic member, wherein the hand-piece is configured to receive the generated high-frequency current and to generate a deep heat using the ceramic member and to apply the deep heat to the abnormal muscle, wherein the hand-piece is configured to generate the deep heat based on a timing schedule set by the control module.

2. The device of claim 1, wherein the display module comprises:
a control panel configured to allow an operator of the device to set timer, a voltage level, and/or an output level including high, middle, low levels;
a LED configured to change its brightness based on the output level and to turn on/off based on a presence/absence of the output; and
an auxiliary power supply configured to supply a power to the control panel and the LED. The device of claim 1, wherein the output adjustment sub-module is configured to generate the output adjustment signal to selectively enable the outputs 70V, 80V, and 98V to be generated respectively for a skin care an-anti-inflammation and a malignant inflammation treatment.

4. The device of claim 1 wherein the device is connected over a wired or wireless network to a computer to allow a remote adjustment of the output and to monitor or regulate the control module.
